(19) United States
(12) Patent Application Publication
(10) Pub. No.: US 2024/0415604 A1
(43) Pub. Date: Dec. 19, 2024

(54) SURGICAL STERILE FIELD

(71) Applicant: SURGICAL DESIGN CORPORATION, Armonk, NY (US)

(72) Inventor: William Banko, Armonk, NY (US)

(73) Assignee: SURGICAL DESIGN CORPORATION, Armonk, NY (US)

(21) Appl. No.: 18/814,059

(22) Filed: Aug. 23, 2024

Related U.S. Application Data
(63) Continuation-in-part of application No. 17/984,780, filed on Nov. 10, 2022.

(51) Int. Cl. A61B 90/40; A61G 10/02

(52) U.S. Cl. CPC A61B 90/40; A61G 10/02; A61B 2090/401

(57) ABSTRACT

A surgical sterile field system includes an expandible flexible enclosure that is transparent in at least part of its expanse and a pair of gloves that are hermetically formed and sealed on a wall of the enclosure to allow access to an inside of the enclosure near a surgical site. Further, the system includes an incision site of a size corresponding to the required surgical site, e.g., the eye, chest or pelvic area. The site has an adhesive at least surrounding its exterior periphery that is attachable to the skin of the patient adjacent the surgical sight and a cover over the incision site adhesive which can be removed prior to use. The enclosure may include a pop-up frame to support it.

### CROSS-REFERENCE TO RELATED PATENT APPLICATIONS

This application is a Continuation-in-part of application Ser. No. 17/984,780, filed Nov. 10, 2022, which is incorporated herein by reference in its entirety.

### FIELD OF THE INVENTION

The present invention relates to a surgical sterile field. More specifically, the present invention relates to a surgical sterile field that is compact, portable, disposable, cost-effective and recyclable.

### BACKGROUND OF THE INVENTION

Surgical procedures performed in a hospital have the benefit of the sterile conditions in hospital operating rooms. These include sterile instruments, surgical drapes, filtered air, as well as gloved and masked surgeons, anesthesiologists, nurses, and operating personnel. The drawback to this hospital procedure is the cost, which is particularly driven by the cost to keep the operating room sterile to protect the patient from infection and the cost of the hospital facilities.

One more recent procedure seeking to reduce the cost of minor surgeries is to carry them out in ambulatory surgery centers. While this reduces the cost of the hospital facility, it still needs to have a sterile operating room and gloved and masked operating personnel, which have costs associated with them. In addition, hospitals may have ordinary rooms dedicated to out-patient surgical procedures that do not require the elaborate sterilizing procedures of operating rooms. These are typically procedures where minor incisions are performed, e.g., the insertion of a cardiac monitor in the chest of a patient, so the patient is less exposed to infections. Nevertheless, the surgeon and other personnel will usually still wear full sterile surgical attire for these procedures.

To further reduce the costs associated with surgeries in hospitals as well as to provide patients with more comfort, an increasing number of physicians have been practicing office-based surgeries, or OBS. OBS are surgical or invasive procedures performed in a location other than a hospital or ambulatory surgery center. In an OBS the procedures, which may include eye surgery (e.g., cataract surgery, retina procedures, refractive lens exchange and oculoplastic surgery), can be performed in a suite located within the physician's office using minimal to moderate anesthetics. However, one major issue in practicing in an OBS is the risk of contamination.

To prevent contamination, the operating room needs to be continuously cleaned and disinfected to create a sterile environment. The surgeon and assistants must scrub their hands meticulously with disinfectant, and wear a sterile gown, gloves, a disposable mask, cap, and disposable shoe coverings. Further, the patient must be extensively covered with sterile drapes. In the case of eye surgery, the eye must be draped with a special sterile cover for intraocular surgery (cataract or vitrectomy). Further, all surgical instruments must be sterile, e.g., by autoclaving them with high pressure steam. These are challenging, especially for those physicians who do not enjoy the same resources as large medical facilities.

Various efforts have been made to help surgeons practice OBS more easily. For example, US Patent Application Publication No. 2015/0238264 of Kerns et al. discloses a sterile ophthalmic surgical drape system that can provide a sterile operating field for ophthalmic medical procedures. The sterile ophthalmic surgical system comprises a drape, a collapsible frame apparatus, a hole of sufficient size for a microscope, a fan, a filter, air directors and slits. The drape system may be configured to be used in conjunction with a surgical tray with presterilized instruments. However, in this drape system, there is no disclosure of how the surgeon can get his hands into the operating site without breaching the sterile environment, except by reverse air flow. Also, the drape does not form a complete sterile enclosure.

Another example is US Patent Application Publication No. 2022/0192780 of Okajima et al., which discloses a portable surgical system including a sterile enclosure and a plurality of sterile sleeves made of a composite-material. However, while it is called “portable,” the surgical system still requires a “rigid frame” structure. Further, in this system, the medical instruments are transferred into the enclosure via material ports that “may be opened and closed by various means, such as zippers, magnetic strips, hook-and loop fasteners.” These ports may cause contamination by introducing non-sterile air full of many particles, bacteria, and viruses, into the container.

The present invention has been made in view of the above-described circumstances to provide a surgical sterile field that is compact and provides an effective sterilized surgical site in a cost-effective manner without relying on a rigid frame structure or requiring heavy equipment such as an electric pump, while providing a sterile way for the surgeon's hands to reach the surgical site.

### SUMMARY OF THE INVENTION

The present invention is directed to a sterile surgical field system that has a flexible enclosure that is transparent in at least part of its expanse and at least one pair of glove chambers that are hermetically formed and sealed on a wall of the enclosure to allow access to an inside of the enclosure near a surgical site. The enclosure is presterilized, e.g., by heat or ultraviolet light. Further, the system includes an incision site of a size corresponding to the required surgical site. The site has an adhesive at least surrounding its exterior periphery that is attachable to the skin of the patient adjacent the surgical sight and a cover over the incision site adhesive which can be removed prior to use. As an alternative, the enclosure at the incision site can be cut by a surgical instrument to expose the surgical site rather than removing the cover.

The flexible enclosure may be inflatable by an external source of sterile air. However, alternatively, and preferably, the enclosure has a collapsible frame within it. A loop attached to an apex of the frame may be pulled away from a base of the frame, so the frame expands creating a vacuum in the enclosure that draws ambient air into the enclosure through a one-way valve with a HEPA filter. As a result, the enclosure is expanded into a bubble shape with a sterile interior.

When used for eye surgery, e.g., cataract removal, it has a transparent portion of the enclosure attached, e.g., by adhesive, to the objective lens of a microscope used by the surgeon during the operation. The system also includes an attachment portion in the form of an eye opening in the enclosure adjacent the patient's eyes, which acts as the surgical site, so it has an adhesive along its outer peripheral edge and an exterior removable cover. A further entrance opening is provided in the enclosure through which cables for powering a surgical instrument such as an ultrasonic phacoemulsification surgical instrument, as well as irrigation and aspiration tubes may pass.

The system is provided at a physician's office with the enclosure collapsed and already sterilized and in a sealed package. If the surgical instrument is of the disposable type, it may be presterilized and provided within the enclosure with its power and fluid tubes partially sealed within a tube in the wall of the enclosure. This will allow the attachment of the fluid lines and power cable to a surgical system outside the enclosure. The transparent portion of the enclosure is fastened to the objective lens of the microscope by the adhesive provided and the enclosure is inflated. During or after expansion or inflation of the enclosure, the eye opening is sealed to the skin of the patient around the eye or eyes. In such a state, the interior of the enclosure is sterile, and the rest of the operating room need not be. The surgeon can put his or her hands in the gloves, pick up the surgical instruments and perform the surgery without contaminating the surgical field. Materials for bandaging the surgical wound that have been presterilized and placed in the enclosure before the procedure can then be used to bandage the patient. In this embodiment everything needed for the procedure is located within the enclosure. At that point, the enclosure can be detached from the microscope and from the skin of the patient. It and its contents, where disposable instruments are used, can then be thrown away.

Having the instruments, medications and bandages sterilized and pre-positioned within the enclosure saves on the preoperative procedures. It is common for the implements, medications (e.g., aesthetics) and materials (e.g., bandages) needed for a procedure, even in an out-patient room, to be bar coded for identification. Before the procedure can begin, the instruments and materials must be gathered, have their bar codes read and placed in a convenient location for the procedure. This setup time is eliminated when everything is located in the enclosure supplied, thus reducing the time the room needs to be occupied. This means that more patients can be seen during the day.

This procedure for eye surgery can be expanded for use in surgical procedures on other parts of the body. In that setting or in eye surgery, additional instruments may be introduced into the sterile enclosure. An instrument tray with the other presterilized instruments may be provided with an adhesive layer over the top of the tray and a cover release layer over the adhesive. The cover layer is removed, and the tray is stuck to the enclosure by the adhesive. To reach the instruments, the surgeon cuts through the enclosure and the adhesive layer.

A particular use of the present invention is in minor surgical procedures such as the implantation of capsules (e.g., therapeutic time release capsules that contain medication (including chemotherapy drugs), hormones or antibiotics), or devices that stimulate or monitor the central or peripheral nervous system, diagnostic monitors, (including cardiac monitors) as well as the insertion of various types of catheters into the patient. See for example, the Adee, “The messy quest to replace drugs with electricity,” MIT Technology Review, May 30, 2024 and the Medtronic Insertable Cardiac Monitor (ICM) https://www.medtronic.com/us-en/healthcare-professionals/products/cardiac-rhythm/cardiac-monitors/reveal-linq-icm/mobile-manager.html. On such catheter is the DxTerity™ diagnostic catheter of Medtronic.

Not only is the sterile surgical field of the present invention useful for surgery in doctor's offices, hospitals and ambulatory surgery centers, but it can also be used for eye surgery or general surgery in disaster relief operations, or a battlefield, or traffic accident cases. In these situations, every minute used in transporting a trauma patient to a hospital can be a matter of life and death. By using the surgical sterile field system of the present invention, procedures like suturing major arteries or veins to stop hemorrhaging can be performed at the disaster location before transporting the patient to the hospital for complete medical care. In addition, since the surgical sterile field system does not require any rigid frame structure, it is easy to carry and is by no means cumbersome.

The microsurgical sterile field embodiment of the present invention provides doctors with significant benefits because of its reduction in cost and infection rates. For those who practice OBS, the microsurgical sterile field of the present invention offers an ideal clinical setting since it makes surgical procedures more efficient, while eliminating all of the time, materials and cumbersome process needed to disinfect the operating room and sterilize the surgical gowns and drapes. The ecological (energy) footprints are also significantly reduced as a result.

### BRIEF DESCRIPTION OF THE DRAWINGS

These and other features and advantages of the present invention will become more readily appreciated when considered in connection with the following detailed description and appended drawings, wherein like designations denote like elements in the various views, and wherein:

FIG. 1 is a side view of a first embodiment of the present invention, in the form of a microsurgical sterile field system for an ophthalmological procedure;

FIG. 2 is a plan view of the elements of the first embodiment of the present invention;

FIG. 3A shows an enlarged view of the entry port and FIG. 3B shows the detailed structure of the entry port for the first embodiment of the present invention;

FIG. 4A is a plan view of the enclosure showing the addition of two types of attachment portions for the first embodiment of the present invention and FIG. 4B is an enlarged side view of the attachment portion formed on the enclosure.

FIG. 5A is a perspective view of an instrument tray used in the surgical sterile field system of first embodiment of the present invention and FIG. 5B is a perspective view of the instrument tray in which the covered layer is peeled to expose the adhesive layer;

FIGS. 6A-6D illustrate the process of making the instruments in the instrument tray accessible to the surgeon in the enclosure of the surgical sterile field system, wherein FIG. 6A shows the cover layers of the attachment portion and the instrument tray removed to expose the adhesive layers, FIG. 6B shows the instrument tray attached to the enclosure; FIG. 6C shows a surgeon cutting the enclosure wall and the tray cover simultaneously, and FIG. 6D shows that the medical instruments are accessible;

FIG. 7 shows a sterile bag that is configured to enclose the instrument tray;

FIG. 8 shows a sterile bag that is configured to enclose the surgical sterile field system of the present invention;

FIG. 9 is a perspective view of a modified version of the first embodiment for use in a general surgical procedure;

FIG. 10 illustrates the process of attaching the attachment portion of the enclosure to the skin of the patient at incision site in general surgery;

FIG. 11 is a second embodiment of the present invention;

FIG. 12 illustrates the work tip of a phacoemulsification hand piece with a laser energy source;

FIG. 13 illustrates a prior art surgical procedure for placing a cardiac monitor in the chest of a patient;

FIG. 14 illustrates a third embodiment of a sterile enclosure according to the present invention that can be used to insert a cardiac monitor in a patient's chest;

FIG. 15 is a plan view of the elements of the third embodiment of the present invention;

FIG. 16 illustrates a fourth embodiment of a sterile enclosure according to the present invention with an inflatable enclosure that can be used in the placement of a cardiac catheter in a pelvic vein of a patient; and

FIG. 17A illustrates the fourth embodiment in which the enclosure has a collapsable frame and FIG. 17B shows a side view of the enclosure of the fourth embodiment where the frame is in a collapsed state.

### DETAILED DESCRIPTION OF THE INVENTION

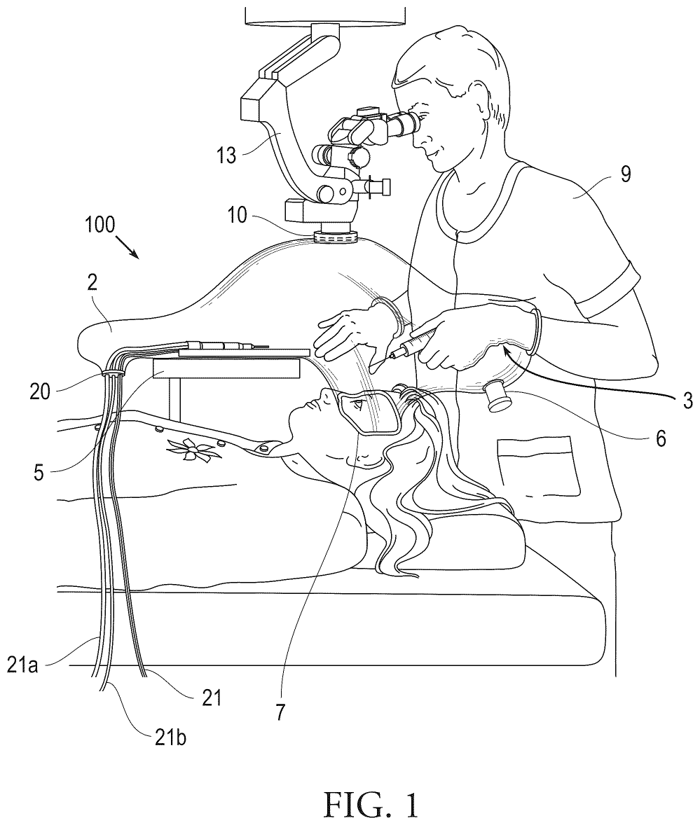

FIG. 1 shows a microsurgical sterile field system 100 of a first embodiment of the present invention for performing an ophthalmological operation. The surgical sterile field system 100 comprises an inflatable enclosure 2 that is made of flexible and inflatable materials, e.g., plastic. It has at least one pair of integral sterile gloves 3 used by a surgeon. The sterile gloves 3 are hermetically sealed to the wall of the enclosure 2 with their rims sealed against the outside environment, but so as to allow for access to the inside of the enclosure 2 by the hands of the surgeon.

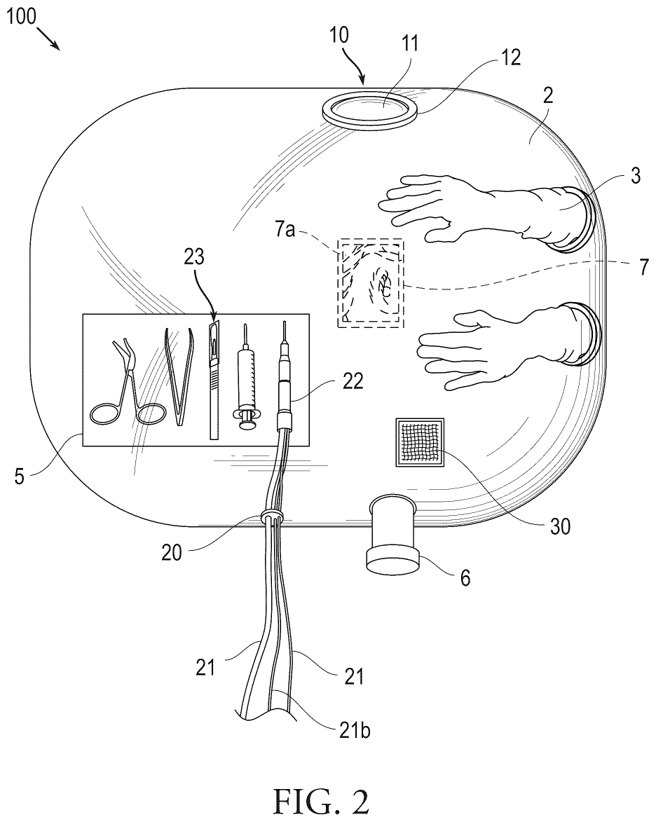

FIG. 1 further shows a surgeon 9 performing an ophthalmological operation while viewing the incision site 7 through a microscope 13 attached to the microscope connection 10. The enclosure 2 is flexible enough to allow the objective lens of the microscope 10 to be aligned with the incision site 7 at the patient's eye. In a particular version, the enclosure may include a clear window 11 at connection 10 to improve the surgeon's view. See FIG. 2.

The microsurgical sterile field system 100 is simple and requires that only a small area be sterile compared to a traditional ophthalmological procedure. For instance, no disposal drapes or gowns are required. Accordingly, the microsurgical sterile field system 100 of the present invention makes it possible to efficiently perform an ophthalmological operation in a cost-effective and environmentally friendly manner, while minimizing the risk of contamination. This includes performing the operation in a doctor's office as opposed to a hospital surgical room or even an OBS suite.

FIG. 2 is a plan view of the enclosure of FIG. 1. It shows the microscope connection 10 and a cable port 20. The microscope connection 10 comprises the view window 11 that is made of clear glass or hard plastic and a rim 12 surrounding the view window 11. The rim 12 is configured to attach to the microscope objective, e.g., by an adhesive. The cable port 20 is formed on the wall of the enclosure 2 so that the power cord 21, irrigation 21a and/or evacuation tubes 21b of a surgical instrument 22 can extend outside of the enclosure 2 through a seal. The surgical instrument and the other instruments 23 needed for the procedure and located in a surgical tray 5 are pre-sterilized and placed inside the enclosure 2 before it is sealed. As can be seen in FIG. 2, there is an incision site 7 that is a portion of the enclosure. It can have adhesive along its periphery 7a that allows it to be fixed to the skin of the patient. A cover is placed over this adhesive during manufacture of the enclosure. At the time of the procedure the cover can be removed, and the enclosure fixed to the skin of the patient, e.g., about the eye as shown in FIG. 2. Once in place, the surgeon can cut through the enclosure with a scalpel on the instrument tray 5 to expose the tissue of the patient where the ocular surgery is to take place.

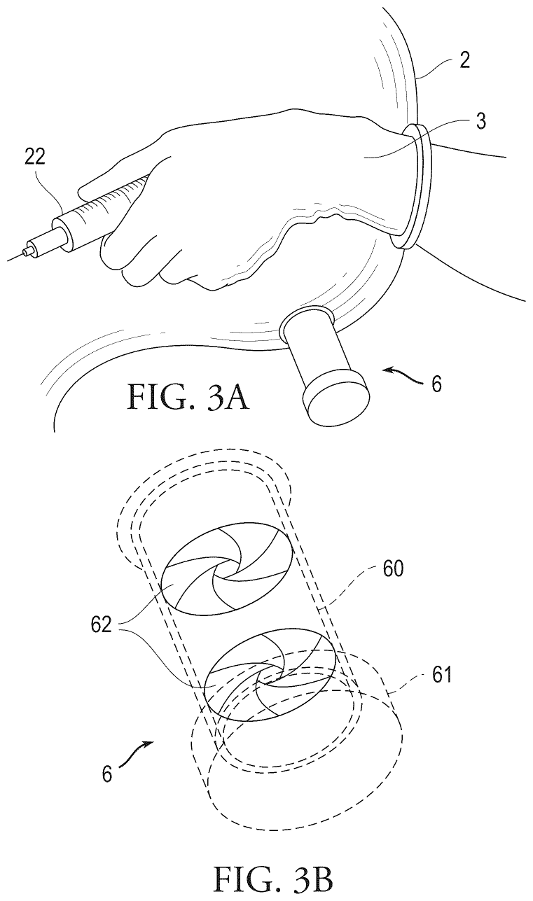

There is a filter 30, such as a HEPA filter, through which air may be drawn into or pushed into the enclosure by a fan or a sterile pump (not shown) to keep the enclosure inflated during the procedure. In one embodiment, the enclosure 2 can be rapidly inflated by using a compressed gas cartridge (not seen) to push air through the filter 30. As an alternative there is an entrance port 6 which is shown enlarged in FIG. 3A that can be used as a way to introduce air into the enclosure from a sterile fan, pump, or gas cartridge. FIG. 3B illustrates the details of one embodiment of the entry port 6 formed on the wall of the enclosure 2. The entry port 6 comprises a tubal body 60, a cap 61, and at least two elastic membranes 62. As shown in FIG. 3B, the elastic membranes 62 are stacked in the tubal body 60 in the longitudinal direction with a certain interval between them in which disinfectant solution or gel is filled. After removing the cap 61, the entry port 6 allows for the entry of a sterilized tube or power cord into the enclosure 2 while minimizing the risk of contamination.

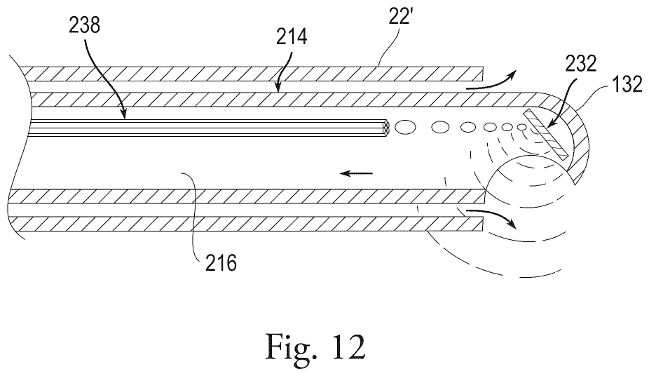

When the enclosure is fully in place and inflated, and the incision site 7 is affixed to the skin of the patient as shown in FIG. 1, the surgeon places his hands in the gloves 3, grasps a scalpel 23 and uses it to remove the portion of the enclosure within the confines of the incision site 7 to expose the underlying tissue of the patient. In a phacoemulsification procedure, an incision is made in the eye by the surgeon to expose the cataract using one of the tools (scalpel) 23 in FIG. 2. Then the surgeon uses the ultrasonic handpiece 22 to break up the cataract while irrigation fluid is bathing the site from irrigation line 21a and the pieces of cataract and excess fluid are drawn away from the area of the cataract by the aspiration line 21b. If needed or desired, the handpiece can use alternatively use laser energy instead of ultrasonic vibration at the tip of a workpiece 22 to soften, breakup and remove a cataract. For example, the laser light source can be a laser in the handpiece 22′ or a fiber-optic cable 238 extending from a laser outside the enclosure to the handpiece 22′ inside the enclosure as shown in FIG. 12. In this arrangement the fiber-optic cable is located inside a tube 132 and directs laser light pulses to a titanium target 232. The effect of the light pulses hitting the target is to generate shockwaves that emulsify the cataract tissue. A channel 214 is formed between the inner surface of the handpiece 22′ and the outer surface of the tube 132 for carrying irrigation fluid to the operating site. Tissue can be aspirated from the site by a suction force applied to the interior 216 of tube 132.

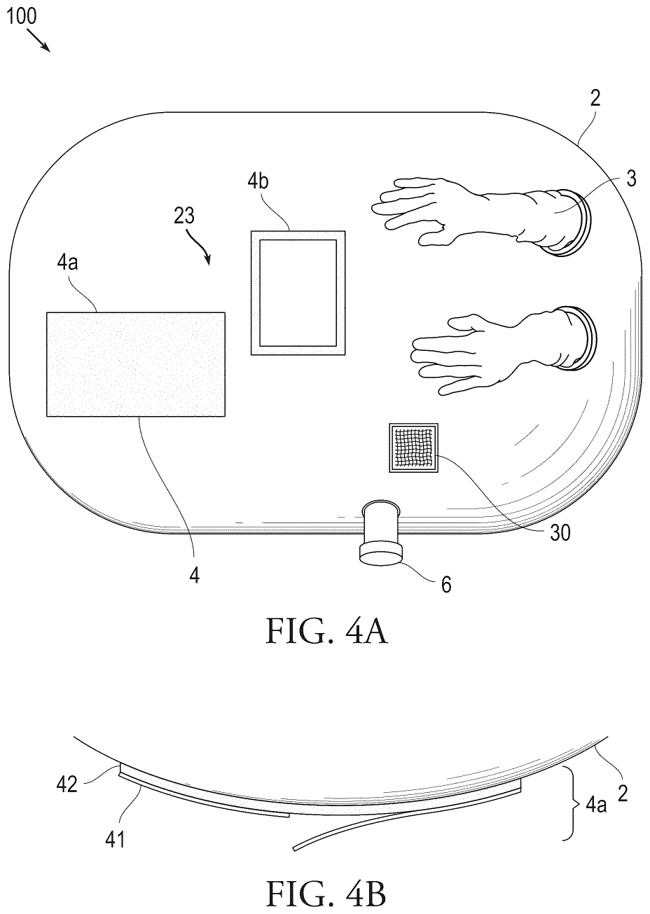

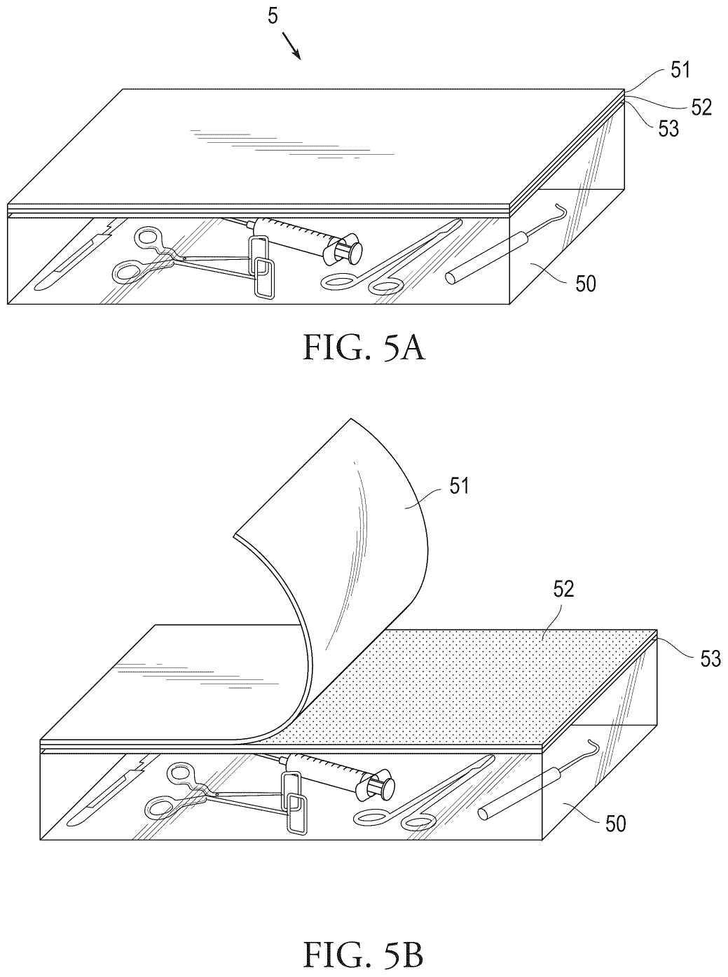

FIG. 4A is a plan view of the surgical sterile field system 100 of the first embodiment. As shown in the drawing, two types of the attachment portions 4 have been formed on the enclosure 2; one 4a is for attaching an instrument tray 5 (FIG. 5A) and another one 4b is for use as an incision site. Although the embodiment shown in FIG. 4A has two attachment portions 4a, 4b, an extra attachment portion 4 may be added if necessary.

As shown in FIG. 4B, the attachment portion 4a has a band-aid type design containing two layers: an adhesive layer 42 disposed on the outer surface of the enclosure 2, and a cover layer 41 disposed on the outer surface of the adhesive layer 42. The cover layer 41 is made of paper or plastic film and can be easily peeled off by hand to expose the adhesive layer 42, to which the instrument tray 5 of FIG. 5A or an incision site on the body of the patient is attached.

As shown in FIGS. 5A & 5B, the instrument tray 5 is composed of a tray box 50 in which sterile medical instruments are store and three layers including a transparent layer 53, an adhesive layer 52, and a cover layer 51 stacked on a top of the instrument tray in this order. Similar to the cover layer 41 of the attachment portion 4 of the enclosure 2, the cover layer 51 of the instrument tray 5 is made of paper or plastic film and can be easily peeled off by hand to expose the adhesive layer 52. The adhesive layer 52 is disposed on the top of the transparent layer 53, which covers the top portion of the tray box 50. In one embodiment, the adhesive layer 52 is also formed transparent.

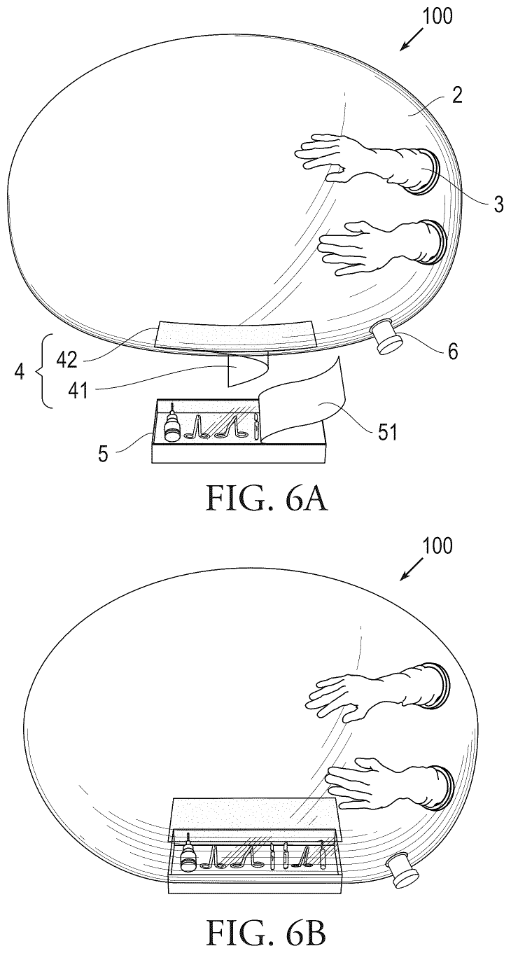

FIGS. 6A-6D illustrate the process of accessing medical instruments in the instrument tray 5 during a surgery. As shown in FIG. 6A, first, the cover layer 41 of the attachment portion 4 of the enclosure 2 and the cover layer 51 of the instrument tray 5 are peeled off. Then, an upper surface of the instrument tray 5 is attached to the adhesive layer 4 of the attachment portion 4 of the enclosure 2. In this embodiment shown in FIG. 6A, the cover layer 41 and the adhesive later 42 of the attachment portion 4 extends along the edges of the attachment portion 4. Likewise, the cover layer 51 and the adhesive layer 52 of the instrument tray 5 at least extend along the edges of the instrument tray 5. Then, the instrument tray 5 is attached to the attachment portion 4 of the enclosure 2 so that the adhesive layer 51 of the instrument tray 5 can be aligned with the adhesive layer 41 of the attachment portion 4.

After attachment, as shown in FIG. 6B, the instrument tray 5 is ready to be accessed by a surgeon. As shown in FIG. 6C, the surgeon inserts his or her hands into the gloves 3 and cuts away the wall of the enclosure 2, the adhesive layer 42 of the attachment portion 4 as well as the adhesive layer 52 and the transparent layer 53 of the instrument tray 5 so that the medical instruments in the instrument tray 5 can be accessed inside the enclosure 2. In some embodiments rather than having the adhesive layer extend along the edges it can cover the entire surface and when access to the tray 5 is needed the surgeon cuts through the layer.

FIG. 6D shows a modified version of the first embodiment where two instrument trays 5, 5′ are attached to the enclosure 2. In this manner, the surgeon can access additional sterilized instruments in the tray 5′ such as scissors, a syringe, a surgical knife and so on while keeping the surgical field sterile. Thus, the surgery can be efficiently performed while reducing the risk of contamination.

The tray 5 shown in FIGS. 5A though 6D can be replaced in a situation where instruments in the tray first supplied are not sufficient for a procedure and additional or other instruments are needed. The method for replacing the existing tray with a new tray is as follows: First, a sterile adhesive patch located in the enclosure is applied over the incision in the transparent layer 53 of the instrument tray. Next, the instrument tray is removed from the exterior of enclosure by detaching it from the attachment portion 4a, thereby exposing the adhesive layer 42 of the attachment portion. The new instrument tray that has different instruments than the instrument tray is provided. Like the original tray it has a transparent layer covering the instruments in the new tray and an adhesive on its top 52 covered by a protective cover 51. Then; the protective cover of the new instrument tray is removed to expose its adhesive layer and the new tray is attached onto the enclosure at the attachment portion by having the adhesive of the new instrument tray contact the adhesive of the attachment portion on the outside of the enclosure, Finally, the surgeon cuts away the transparent layer 53 of the new instrument tray so that the different medical instruments in the instrument tray can be exposed inside the enclosure.

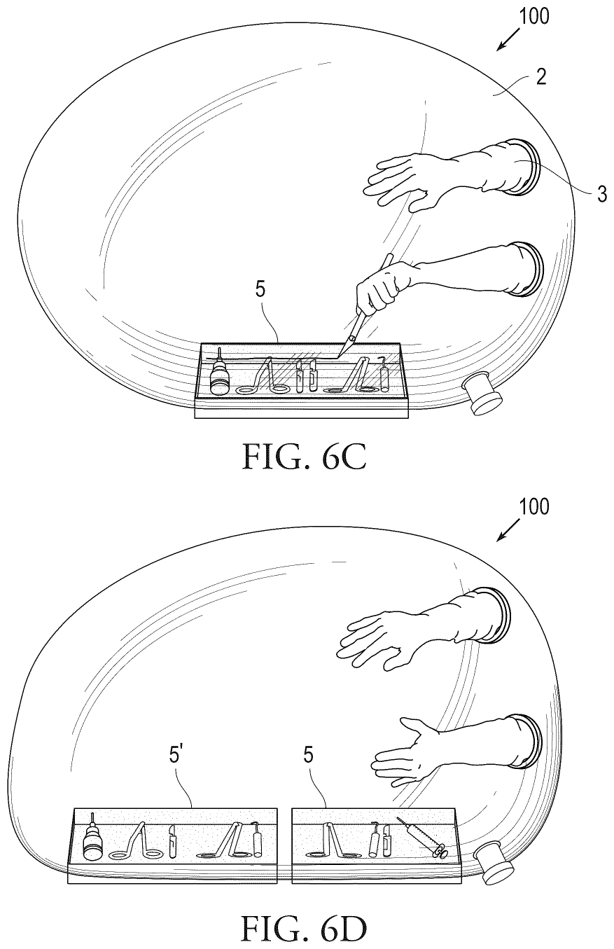

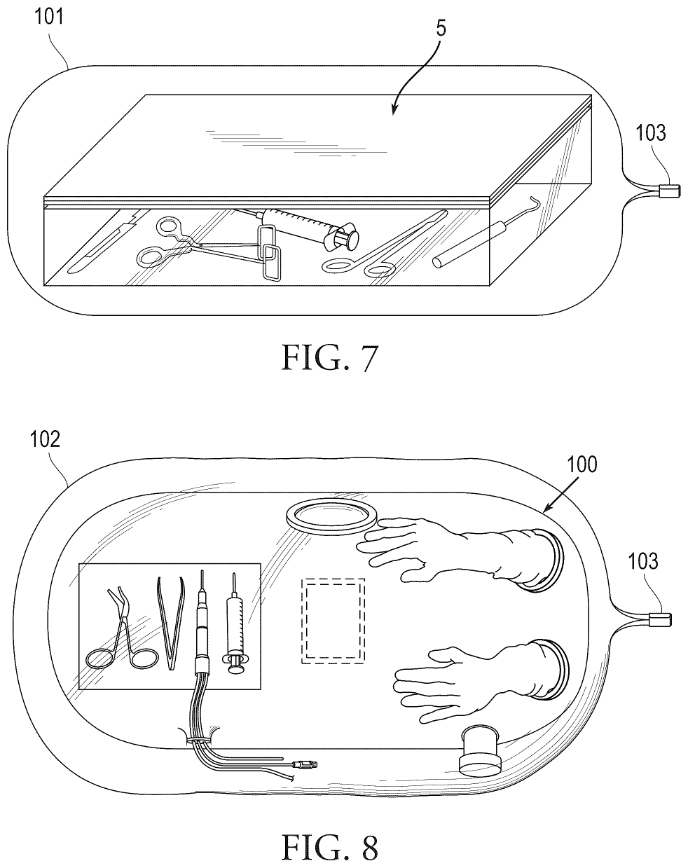

In the present invention, a sterile bag is utilized to store sterilized components. For instance, as shown in FIG. 7, a small sterile bag 101 is used for containing relatively small components such as the instrument tray 5. The sterile bags 101 must be removed before accessing the instrument tray 5. FIG. 8 shows a large sterile bag 102 that is used for containing the entire surgical sterile field system 100. The sterile bags 101, 102 have an opening port 103 that can be pulled apart to open the sterile bags 101, 102.

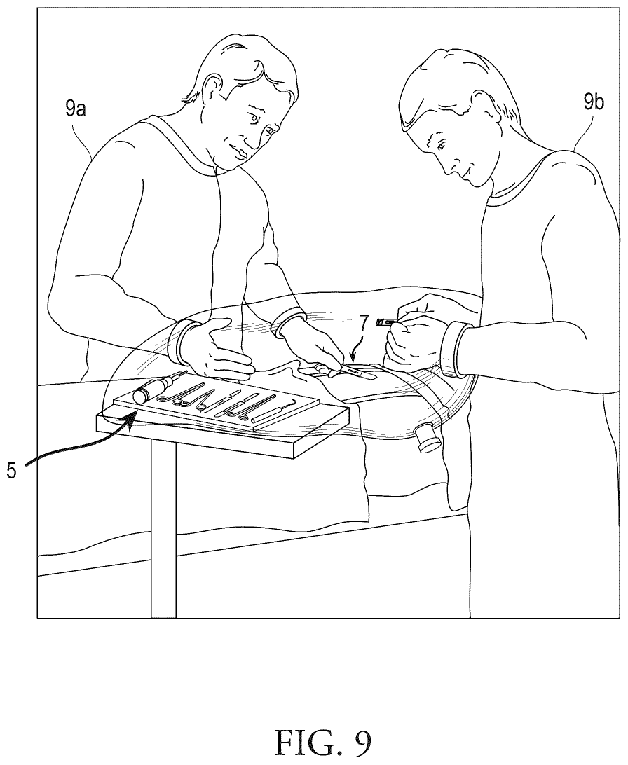

FIG. 9 shows a modified version of the first embodiment where two surgeons 9a, 9b perform general surgery. The surgery may be performed in a hospital or an OBS or a doctor's office using the surgical sterile field system 100 of the present invention. However, the location is not limited to an office, but may include an open field such as a battlefield or an accident site. No matter where the operation is performed, thanks to the surgical sterile field system 100, the patient is sufficiently protected from contamination.

As seen in FIG. 9, two pairs of the integral gloves are provided on opposite sides of the wall of the enclosure 2. Using these gloves, the surgeons have access to the surgical tools in tray or box 5. In this case the surgery is performed without a microscope. In addition, the incision site 7 can by anywhere on the body of the patient. The shape of the enclosure may be folded or otherwise adjusted (e.g., changed in size or shape) to fit the part of the body that is being operated on.

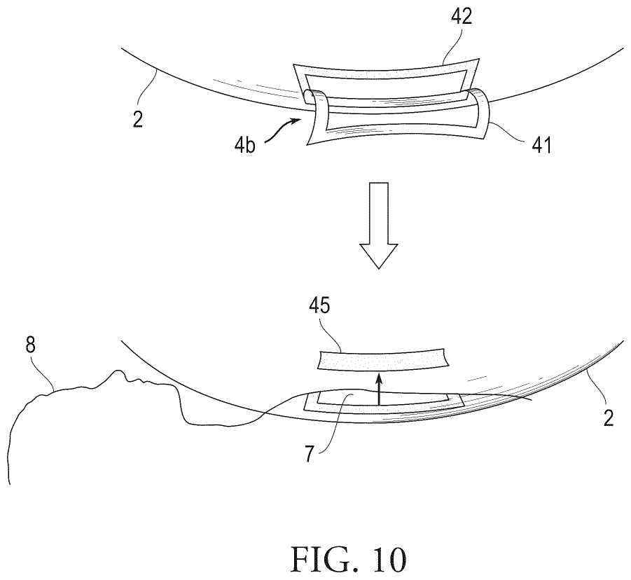

FIG. 10 illustrates the process of attaching the enclosure 2 to the incision site 7. Similar to the process described above, first the cover layer 41 of the attachment portion of the enclosure 2 is peeled off. Then, the enclosure 2 is lowered so that the adhesive layer of the attachment portion is placed over the incision site 7 of the patient 8. Then, the enclosure 2 is attached directly to the area around the incision site 7. After attachment, the surgeon can simultaneously cut away and remove a portion 45 of the wall of the enclosure 2 and a part of the attachment portion 4 that is surrounded by the adhesive layer 42 so that the incision site 7 is accessible. The removed portion of the enclosure 2 and the attachment portion 4 may be left inside the enclosure 2. In this manner, the surgeon can perform a surgery while minimizing the risk of contamination to the incision site 7. When performing ocular surgery, it is preferrable that the adhesive extend about the periphery of the incision site. In general surgery, e.g., chest surgery as shown in FIG. 10, it may be preferable to have the adhesive extend over the entire area and be sealed to the patient's chest. Rather than cut out the portion 45 the surgeon can leave it in place and make an incision directly through the enclosure and adhesive at the incision cite. Once the operation is complete, the wound can even be sutured while the adhesive layer is still adhering to the patient's chest. In such a situation it is beneficial to include an antibiotic or other medicine in the adhesive to guard against infection and to promote healing.

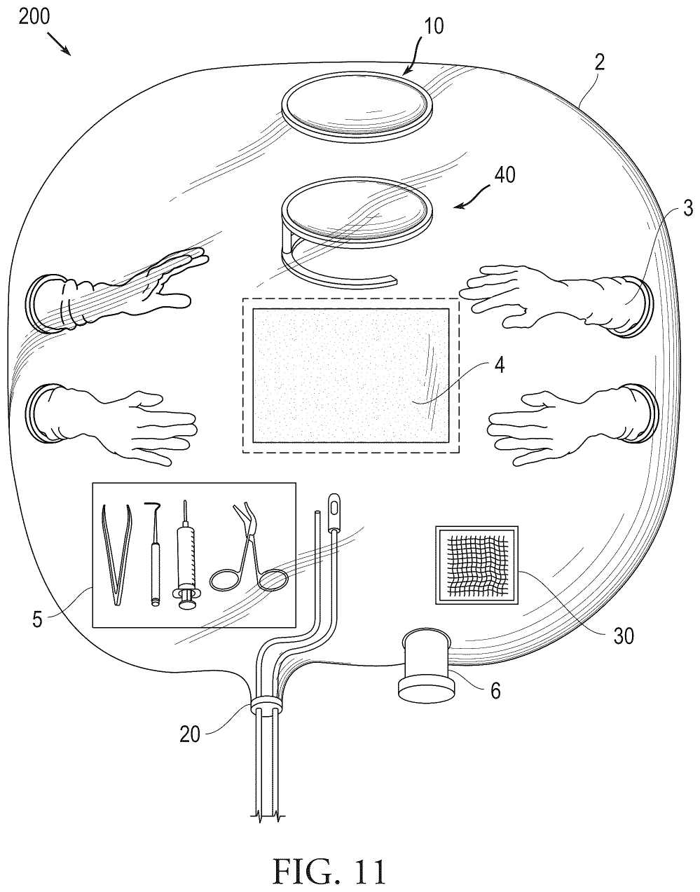

FIG. 11 is a second embodiment of the present invention. In this embodiment the microsurgical sterile field system 200 differs from the system 100 of the first embodiment in the presence of an internal magnifying glass 40. The magnifying glass 40 is aligned with the incision site 7 and configured to magnify the incision site 7. Further, there can be a modified version of the second embodiment where the entry port 6 for the enclosure is omitted.

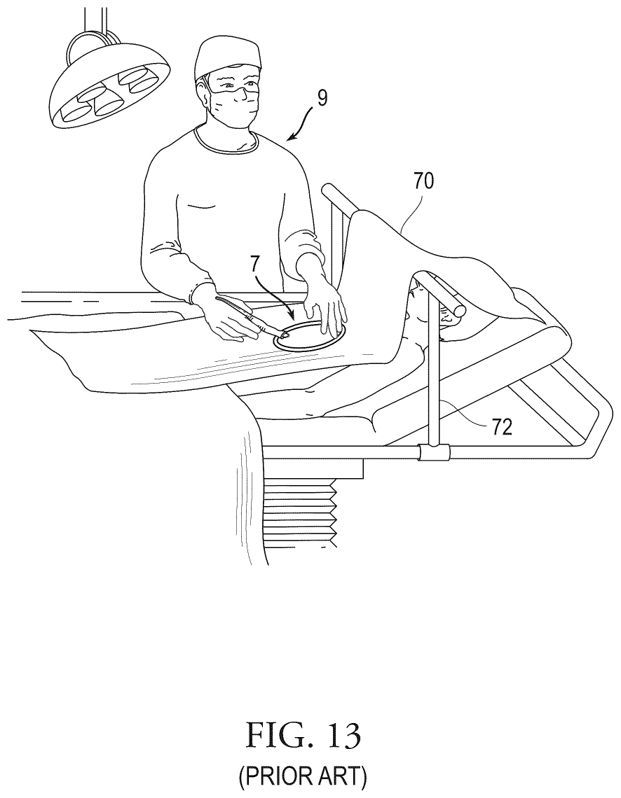

FIG. 13 shows a prior setup for inserting a medical device, for example an insertable cardiac monitor, or medicine in a cavity formed in the body of a patient, e.g., in the patient's chest. In this out-patient setting, the surgeon 9 wears a mask, gloves, and a gown. A drape is 70 placed over the patient. It can pass over a frame 72 that holds it away from the patient's face so they can breathe, but not on the surgical site. Nevertheless, having the drape over the patient's face is uncomfortable for the patient. A hole is provided in the drape at the location of the surgical site 7.

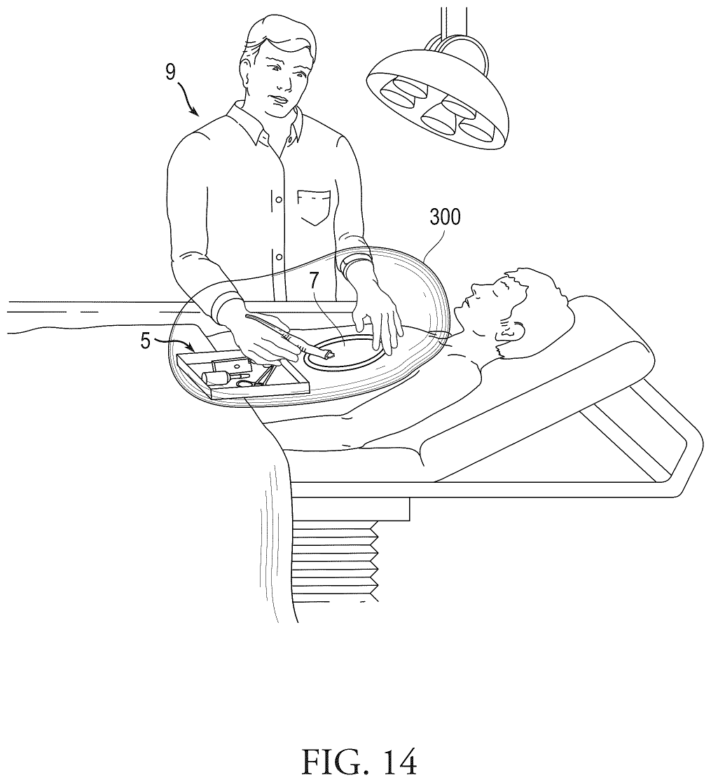

An alternative arrangement for conducting the surgery depicted in FIG. 13 is shown in FIG. 14, which represents a fourth embodiment of the present invention. In the arrangement of FIG. 14, an expanded or inflated transparent enclosure 300 is located over the surgical site 7 in the manner discussed above. Within the enclosure 300 there is a box 5 with surgical instruments that allow the surgeon to make an incision in the chest of the patient. This incision can be used to insert capsules, e.g., therapeutic time release capsules and cardiac monitors such as those sold by Medtronic, in the chest of a patient. In the case of the cardiac monitor, the instruments include an insertion tool loaded with the cardiac monitor and an incision tool in the form of a handle with a scalpel tip at its end as shown in the hands of the surgeon in FIG. 14. The technique for insertion of the monitor is shown on the Medtronic website. However, this procedure can be conducted in an ordinary room without special sterilization and filtering. Further, the surgeon need not wear a mask, glove, and gown. Nevertheless, the surgical site 7 is protected from infectious materials, while allowing the patient to breathe normally without a drape over his face. As shown by the manufacturer of the Medtronic device, insertion is suggested merely with a topical disinfectant spread to the insertion site. This would not prove the type of significantly added protection from infection provided by the present invention.

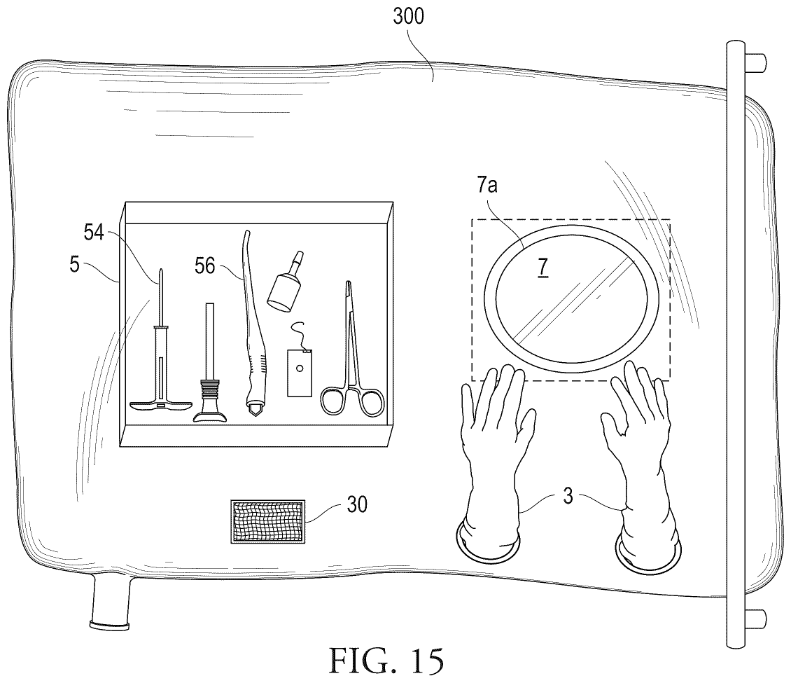

As shown in FIG. 15, which is a plan view of the enclosure 300 of FIG. 14, the surgical instrument and other instruments needed for the procedure are pre-sterilized and located in the surgical box or tray 5 within the enclosure 300. Typical instruments for implantation in the chest wall are the inserter 54 and an incision tool 56 for creating an opening in the skin of the chest. As can be seen there is an incision site 7 that is a portion of the enclosure, and it can have adhesive along its periphery 7a that allows it to be fixed to the skin of the patient. Once in place, the surgeon can cut through the enclosure with a scalpel from the instrument tray 5 to expose the tissue of the patient where the chest insertion is to take place. There is a filter 30, such as a HEPA filter, through which air may be drawn into or pushed into the enclosure by a fan or a sterile pump (not shown) to keep the enclosure inflated during the procedure.

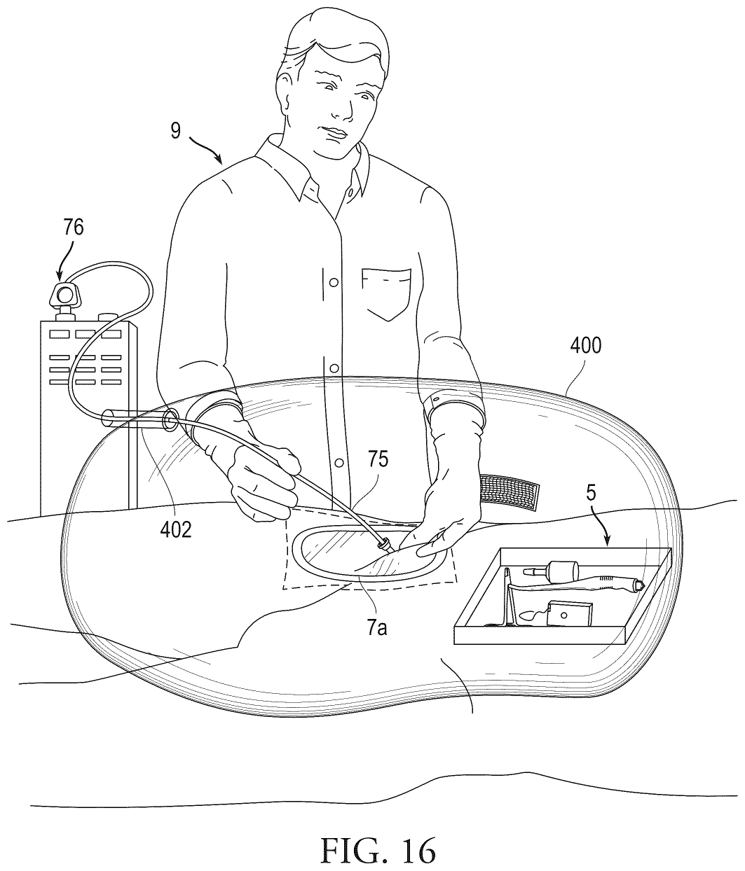

A fourth embodiment is shown in FIG. 16. In that figure an enclosure 400 is used to isolate the pelvic area of a patient undergoing a catheter insertion procedure, where a cardiac catheter 75 is placed in a pelvic vein. The catheter enters the enclosure 400 through a sealed tube 402. This tube allows the catheter to be moved into or withdrawn from the enclosure without allowing surrounding atmosphere to enter the enclosure. Thus, it may be similar to or the same as port 20 in FIG. 2. Once the catheter is placed in the vein it is pushed through the vein to the heart. A machine 76 is connected to the end of catheter outside of the enclosure and provides the capability for the catheter to carry out its prescribed function, e.g., to expand in a clogged artery when the catheter carries a stent. The enclosure 400 has an adhesive along its base which is covered by a release strip. When ready for use the release strip is removed and the enclosure is fastened to abdomen and upper thighs of the patient completely covering the pelvic areas surrounding the surgical site.

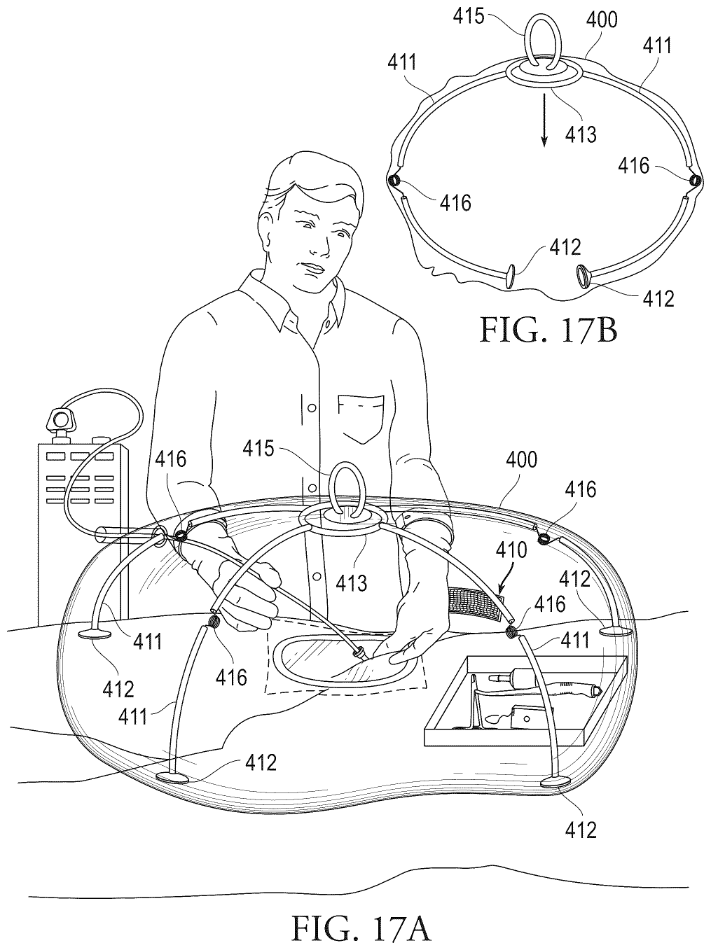

In the arrangement of FIG. 17A the enclosure the enclosure 400 of FIG. 16 further includes a frame 410 to keep the enclosure in an expanded shape. This frame can be used in addition to air inflation of the enclosure or in place of it. If force air is not needed or desired, certain benefits are achieved. First, the cost of the equipment for air inflation is eliminated. This includes a fan and any filter system. Second, the noise of the fan is eliminated. Third, the possibility is removed that the air insertion equipment will block the view of the patient by the surgeon.

Preferably the frame 410 is made from flexible spokes 411 with feet 412 at one end. The other ends of the spokes are attached to an apical ring or disk 413 at the top of the frame. The frame parts can be thin flexible plastic or spring metal rods. In one version the frame is made collapsible as shown in FIG. 17B. To assist in making the frame collapsible, spring-loaded hinged joints 416 are provided in each of the spokes of the frame. FIG. 17B shows the frame in its collapsed form. The enclosure 400 is also collapsed when the frame is collapsed. It can be stacked and shipped in this collapsed shape after its interior has been sterilized. When it is time to use the enclosure 400, the surgeon can pull up on a loop 415, allowing the springs in the hinged joints to cause the frame to pop-up into its expanded shape.

According to the surgical sterile field system of the present invention, it is possible to improve the efficiency of the creation of a sterile surgical site so that surgical procedures can be performed in a doctor's office. The surgery can be in different parts of the body, e.g., the eye, the chest and the pelvic area. Further, it is possible to suppress the cost associated with the procedures since the surgical sterile field system of the present invention does not require a significant amount of sterilization of the operating site. In addition, it is possible to reduce the risk of contamination that may occur in out-patent and OBS procedure rooms. Therefore, the present invention has industrial benefit and applicability.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description and is only limited by the scope of the appended claims.

1. A surgical sterile field system comprising:
an expandable flexible enclosure that is transparent in at least part of its expanse;
a pair of gloves that are hermetically formed and sealed on a wall of the enclosure to allow access by a user's hands to an inside of the enclosure near a surgical site; and
an incision site of a size corresponding to the required surgical area, said incision site having an adhesive covering at least its exterior periphery, which adhesive is attachable to the skin of the patient adjacent the surgical site, and a peelable cover over the adhesive at the incision site that guards against contact with the adhesive until placed on the patient, wherein upon removal of the cover and application of the incision site of the enclosure to the patient, the enclosure can be cut by a surgical instrument to expose the surgical site while maintaining a sterile environment at the surgical site.

2. The surgical sterile field system according to claim 1, further including
at least one attachment portion formed on an outer surface of the wall of the enclosure,
wherein the attachment portion is composed of an adhesive layer and a protective cover layer stacked on the adhesive layer, and
the cover layer can be manually removed from the adhesive layer.

3. The surgical sterile field system according to claim 1, wherein the enclosure has a filter through which sterilized air is supplied to expand the enclosure by inflation.

4. The surgical sterile field system according to claim 2, further comprising:
an instrument tray that is configured to contain sterilized medical instruments, the instrument tray having a transparent layer covering the instruments, an adhesive on its top and a protective cover over its adhesive;
wherein the at least one attachment portion is configured to attach to the top of the instrument tray after its protective cover has been removed.

5. The surgical sterile field system according to claim 1 wherein all of the instruments, medications and bandage materials for a procedure are supplied inside the enclosure.

6. The surgical sterile field system according to claim 1, wherein the enclosure and the adhesive covering are made of plastic that can be penetrated by from inside the enclosure by a surgical knife.

7. The surgical sterile field system according to claim 1, wherein the enclosure is adapted to have its incision site surround a surgical area on the chest of the patient.

8. The surgical sterile field system according to claim 5, wherein the enclosure is adapted to have its incision site surround a surgical area on the chest of the patient and the sterilized medical instruments in the instrument tray include a diagnostic monitor, an incision tool for creating an opening in the skin of the chest, and an insertion tool for placing the monitor in the opening in the skin.

9. The surgical sterile field system according to claim 8 wherein the diagnostic monitor is a cardiac monitor.

10. The surgical sterile field system according to claim 7 further including an instrument tray that is configured to contain sterilized medical instruments, and the sterilized medical instruments in the instrument tray include a time release capsule, an incision tool for creating an opening in the skin of the patient, and an insertion tool for placing the capsule in the opening in the skin.

11. The surgical sterile field system according to claim 10 wherein the time release capsule contains at least one of medication, hormones and antibiotics.

12. The surgical sterile field system according to claim 11 wherein the medication is a chemotherapy drug for treatment of cancer.

13. The surgical sterile field system according to claim 1 wherein the enclosure is adapted to have its incision site surround a surgical area providing access to the nervous system of the patient and further including an instrument tray that is configured to contain sterilized medical instruments, where the sterilized medical instruments in the instrument tray include a nervous system stimulator, an incision tool for creating an opening in the skin of the patient, and an insertion tool for placing the stimulator in the opening in the skin.

14. The surgical sterile field system according to claim 1 wherein the enclosure is adapted to have its incision site surround a surgical area providing access to the venous system of the patient and further including an instrument tray that is configured to contain sterilized medical instruments, where the sterilized medical instruments in the instrument tray include a catheter, an incision tool for creating an opening in the skin of the patient, and an insertion tool for placing the catheter in the opening in the skin.

15. The surgical sterile field system according to claim 14 wherein the catheter is self is self contained.

16. The surgical sterile field system according to claim 14 wherein the enclosure further includes a sealed tube extending through the wall on the enclosure and an end of the catheter passes through the sealed tube to a device outside of the enclosure.

17. The surgical sterile field system according to claim 1, wherein the enclosure is adapted to have its incision site surround a surgical area in the pelvic region of the patient.

18. The surgical sterile field system according to claim 5, wherein the enclosure is adapted to have its incision site surround a surgical area in the pelvic region of the patient and one of the instruments is a catheter.

19. The surgical sterile field system according to claim 5, wherein the enclosure is adapted to have its incision site surround a surgical area in the pelvic region of the patient and the sterilized medical instruments in the instrument tray include an incision tool; and
wherein the enclosure further includes a sealed tube passing through the wall on the enclosure and allowing passage of a catheter into the enclosure for insertion into a pelvic vein of the patient with the incision tool.

20. The surgical sterile filed system according to claim 1 wherein the enclosure is adapted to have its incision site surround a surgical area in the abdominal region of the patient.

21. The surgical sterile filed system according to claim 1 wherein the enclosure is adapted to have its incision site surround a surgical area on the back region of the patient.

22. The surgical sterile filed system according to claim 1 wherein the enclosure is adapted to have its incision site surround a surgical area on one of the arm, leg or neck region of the patient.

23. The surgical sterile field system according to claim 1 further including a flexible frame within the enclosure for supporting the enclosure in an expanded state, while allowing the enclosure to collapse by pressure on the frame.

24. The surgical sterile field system according to claim 23 wherein the flexible frame comprises a plurality of spaced flexible rods with feet at one end, the other ends of the rods being attached to an apical ring at the top of the frame.

25. The surgical sterile field system according to claim 23 wherein the rods are made of flexible plastics.

26. The surgical sterile field system according to claim 23 wherein the rods are made of spring metal.

27. The surgical sterile field system according to claim 23 wherein the flexible rods each includes a spring-loaded hinged joint which allow the rod to fold onto itself as a force is applied down on the apical ring of the frame to collapse the enclosure, and whose spring action helps the enclosure to expand when a force pulls up on the apical ring.

28. The surgical sterile field system according to claim 23 arranged so that the enclosure and frame can be collapsed for stacking and shipping.

29. The surgical sterile field system according to claim 24 wherein the interior of the enclosure is sterilized prior to stacking and shipping.

30. The surgical sterile field system according to claim 23 further including a loop attached to the apical ring that can be grabbed by a user to assist in applying a pull force on the enclosure to cause it to expand.

31. The surgical sterile field system according to claim 23 further including a loop attached to the apical ring that can be grabbed by a user to assist in applying a pull force on the enclosure to cause it to expand and the springs in the hinged joints to cause the frame to pop-up into its expanded shape.

32. A method of performing a surgery using the surgical sterile field system according to claim 4, the method comprising the steps of:
inflating the enclosure to expand it by supplying sterilized air into the enclosure;
removing the cover layer of the attachment portion to expose the adhesive layer thereof;
removing the cover layer of the instrument tray to expose the adhesive layer thereof;
attaching the instrument tray to the enclosure; and
simultaneously cutting away the wall of the enclosure together with the adhesive layer of the attachment portion and the adhesive layer and the transparent layer of the instrument tray so that the medical instruments in the instrument tray can be exposed inside the enclosure.

33. The method of claim 32 in which a tray is replaced with a new tray and further including the steps of:
applying a sterile adhesive patch located in the enclosure over the incision in the transparent layer of the instrument tray;
removing the instrument tray from the exterior of enclosure by detaching it from the attachment portion, thereby exposing the adhesive layer of the attachment portion;
providing a new instrument tray that has different instruments than the instrument tray, a transparent layer covering instruments in the new tray and an adhesive on its top covered by a protective cover;
removing the protective cover of the new instrument tray to expose its adhesive layer;
attaching the new instrument tray onto the enclosure at the attachment portion by having the adhesive of the new instrument tray contact the adhesive of the attachment portion on the outside of the enclosure; and
cutting away the transparent layer of the new instrument tray so that the different medical instruments in the instrument tray can be exposed inside the enclosure.
